SEQUENCE LISTING (12 sequences)

SEQ ID NO: 1 - DNA, 30 bp, Artificial Sequence
  Description of Artificial Sequence Synthetic primer
ccttttatcgatccatgatggttctgcagg

SEQ ID NO: 2 - DNA, 34 bp, Artificial Sequence
  Description of Artificial Sequence Synthetic primer
ttaaaagtcgacttacctgtttatgcagatcctc

SEQ ID NO: 3 - DNA, 31 bp, Artificial Sequence
  Description of Artificial Sequence Synthetic primer
ccttttatcgatcaacatgcgccctgaagac

SEQ ID NO: 4 - DNA, 45 bp, Artificial Sequence
  Description of Artificial Sequence Synthetic primer
ttaaaactcgaggtgtgagcacgtaccgttggtggcctgagtgtg

SEQ ID NO: 5 - DNA, 17 bp, Artificial Sequence
  Description of Artificial Sequence Synthetic primer
ggattggaaagaggctc

SEQ ID NO: 6 - DNA, 17 bp, Artificial Sequence
  Description of Artificial Sequence Synthetic primer
gcactgcccgcttctcc

SEQ ID NO: 7 - DNA, 22 bp, Artificial Sequence
  Description of Artificial Sequence Synthetic primer
taatacaaagtctgcagctggc

SEQ ID NO: 8 - DNA, 17 bp, Artificial Sequence
  Description of Artificial Sequence Synthetic primer
agcaatgttagccagcc

SEQ ID NO: 9 - DNA, 22 bp, Artificial Sequence
  Description of Artificial Sequence Synthetic primer
taatacaaagtctgcagctggc

SEQ ID NO: 10 - DNA, 17 bp, Artificial Sequence
  Description of Artificial Sequence Synthetic primer
agcaatgttagccagcc

SEQ ID NO: 11 - DNA, 17 bp, Artificial Sequence
  Description of Artificial Sequence Synthetic primer
ggattggaaagaggctc

SEQ ID NO: 12 - DNA, 17 bp, Artificial Sequence
  Description of Artificial Sequence Synthetic primer
gcactgcccgcttctcc

(19) United States
(12) Patent Application Publication
(10) Pub. No.: US 2010/0138936 A1
(43) Pub. Date: Jun. 3, 2010

(54) Transgenic Mice and Use Thereof as an Experimental Model

(76) Inventors: Yu-Chun Lone, Paris (FR); Anthony Pajot, Paris (FR); François Lemonnier, Bourg La Reine (FR)

Correspondence Address: NIXON & VANDERHYE, PC, 901 NORTH GLEBE ROAD, 11TH FLOOR, ARLINGTON, VA, 22203, US

(21) Appl. No.: 11/885,429

(22) PCT Filed: Mar. 6, 2006
(86) PCT No.: PCT/FR2006/000502

§ 371 Date: Dec. 4, 2007

(30) Foreign Application Priority Data
Mar. 4, 2005 (FR) 0502211

(51) Int. Cl. C12N 15/06; C12N 15/85; A01K 67/027; G01N 33/00; C12N 5/078; C12N 5/18

(52) U.S. Cl. 800/3; 800/21; 800/18; 435/355; 435/452; 435/326

(57) ABSTRACT

The invention relates to the use of an expression vector construction coding for the functional HLA-DPal03β401 complex specifically identified by anti-HLA-DP antibodies, in order to create transgenic mice. The invention also relates to the use of the transgenic mice obtained, such as for the comparative preclinical study of the efficacy of vaccine candidates in order to asses the risks associated with the unwanted induction of an autoimmune disease and in order to determine a therapeutic strategy.

The invention relates to the creation of novel HLA class II transgenic mice and to the use thereof as an animal experimental model for studying the effectiveness of vaccine candidates.

At the current time, there is an urgent need for effective vaccines against infections with viruses responsible for serious pathologies such as, for example, HIV, HCV, CMV or HSV.

In order for a vaccine to exert a protective effect in the majority of immunized individuals, the vaccinal protection should jointly mobilize the CD4+ T lymphocytes and the CD8+ T lymphocytes specific for the virus, for rapid elimination of the infected cells, and also the B lymphocytes producing the neutralizing antivirus antibodies, in order to prevent reinfection with the same virus.

All these effects provide a very effective overall protection and memory.

The virus-specific T lymphocytes recognize the peptides derived from certain proteins of the virus, presented by the HLA class I and class II molecules of the infected cells.

Although numerous peptides presented by HLA class I molecules have been identified, on the other hand, those presented by HLA class II molecules are relatively unknown. The identification of these peptides is, however, determining for the production of an effective vaccine.

The study of these regulatory responses using human material is difficult due to the polymorphism of HLA class II molecules and the expression of numerous HLA class II molecules in the same individual.

HLA class II transgenic mice are currently available. These mice express either an isolated HLA-DRα chain which pairs with the mouse IEβ^{b} chain, or a pair of α and β chains of human origin.

Hagihara et al. have thus produced mice transgenic for wild-type HLA-DPβ401α01 in the context of xenoreaction studies. However, the reactions observed appear to be very weak. In fact, no detailed study has been provided on the functionality of the transgene and its involvement in the education that results in a normal immune response capacity in these mice, thereby greatly suggesting that these mice are not functional.

More recently, HLA class II transgenic mice which are H-2 class II KO through inactivation in the H-2^{b} haplotype of the IAβ chain have been created in order to study autoimmune diseases (see Taneja and David, 2000, Arthritis Res., 2, 205-7). Some of these mice have made it possible to validate parasite CD4 epitopes in vivo.

The inventors' studies in this field have led them to develop a novel animal experimental model of humanized mice transgenic for HLA-DP and with a knockout for the H-2 class II genes, using a specific allele represented at a rate of approximately 50% in the Caucasian population, thereby making it possible to search for a vaccine with a better coverage of the population compared with the models available to date.

These mice have proved to be of great value for the comparative preclinical study of the efficacy of various vaccine candidates and for assessing the risks associated with an unwanted induction of an autoimmune disease. They also make it possible to determine the best therapeutic strategy to be adopted.

The aim of the invention is therefore the use of expression vectors encoding functional complexes specifically recognized by anti-HLA-DP antibodies in order to create transgenic mice.

The aim of the invention is also to provide such mice and their use as an experimental model for studying the immune responses induced after viral infection or by the compounds tested.

The invention thus relates to the use, in order to create transgenic mice, of an expression vector construct encoding the functional HLA-DPα103β401 complex, specifically recognized by anti-HLA-DP antibodies, characterized in that the construct comprises murine promoters of the IAβ and IAα genes associating the cDNAs encoding DPα103 and DPβ401.

The transgenic mice of the invention are characterized in that they are humanized mice expressing individually the HLA-DPα103 or HLA-DPβ401 transgene, or simultaneously the 2 transgenes.

The invention is in particular related to mice transgenic for HLA-DP(α103β401) and knockout for H-2 class II (HLA-DP+/+IAβ°/°).

These mice are advantageously obtained by microinjection into the oocytes of founder mice of DNA fragments, encoding said complex, obtained from plasmids containing them, crossing between the founder mice expressing individually the HLA-DPα103 or HLA-DPβ401 transgene, and crossing the mice expressing simultaneously the two transgenes with IAβ° mice.

Mice particularly preferred for their high capacity for response to epitopes are also transgenic for hCD₄ and knockout for mCD₄. They are HLA-DP(α103β401)IAβ°.hCD₄^{+/+}mCD₄°/° mice.

In order to create HLA-DP(α103β401)IAβ°.hCD₄^{+/+}mCD₄^{°/°} mice, the transgenic mice of the invention (HLA-DP+/+IAβ°/°) are crossed successively with hCD₄^{+/+} mice and mCD₄°/° mice, so as to obtain a novel mouse corresponding to the HLA-DP(α103β401)IAβ°.hCD₄^{+/+}mCD₄°/° phenotypes.

The mice of the invention (HLA-DP(α103β401)IAβ° and HLA-DP(α103β401)IAβ°.hCD₄^{+/+}mCD₄°/°) represent novel animal experimental models of humanized mice for identifying HLA-DP restricted epitopes which are immunogenic in mice and in humans.

The epitopes thus identified can then be used as subunits for the development of vaccines, in particular polyepitope vaccines.

They make it possible to develop novel reagents that can be used for monitoring the specific immune response post-infection or post-immunization (for example, peptide/HLA class II tetramers).

They can also be used for developing tests to predict the state of protection of an individual with respect to a given virus and/or monitoring a viral infection.

The novel experimental model of the invention also makes it possible advantageously to compare the effectiveness of the T helper response induced by various vaccine candidates or various immunization protocols. In this respect, they represent a model for assessing the risks of autoimmune disease induction.

The novel experimental model of the invention can also be exploited for analyzing the cellular cooperation between CD4 T lymphocytes and CD8 T lymphocytes in mice, following an immunization against a viral antigen, and determining, if desired, the pairs of HTL and CTL epitopes acting in synergy.

The invention also relates to the CD4+ lymphocytes isolated after immunization of a transgenic mouse as defined above.

It also relates to the use of these lymphocytes for establishing specific cell lines and for creating T-specific hybridomas by cell fusion, according to conventional techniques, these cell lines and these hybridomas also being targeted by the invention as novel products.

These cell lines are of great advantage for the production of the specific monitoring reagents, for example for calibrating or carrying out HLA class II tetramer quality tests.

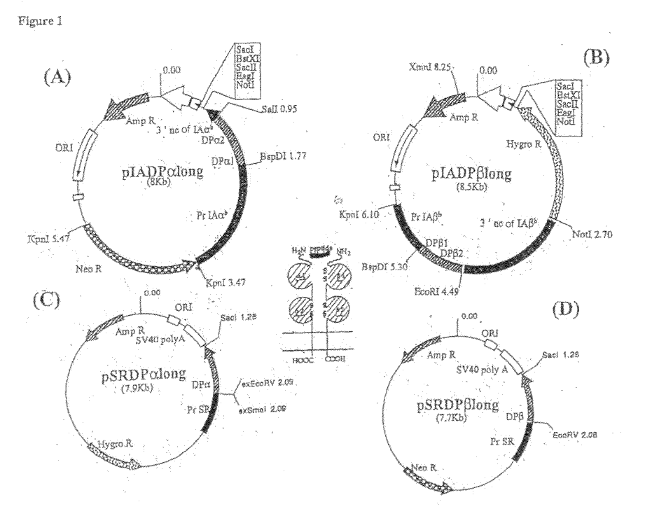

Other characteristics and advantages of the invention are given in the examples which follow, in which reference is made to FIGS. 1 to 10, which represent, respectively:

FIG. 1, the maps of 4 plasmid constructs used according to the invention;

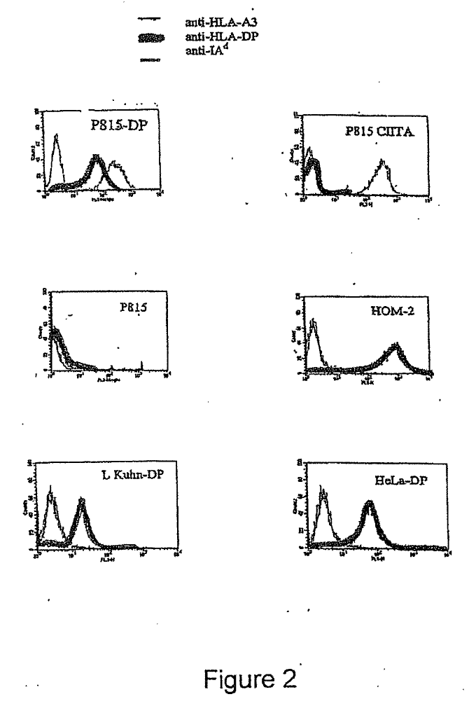

FIG. 2, the assessment of the functionality of two vectors used according to the invention;

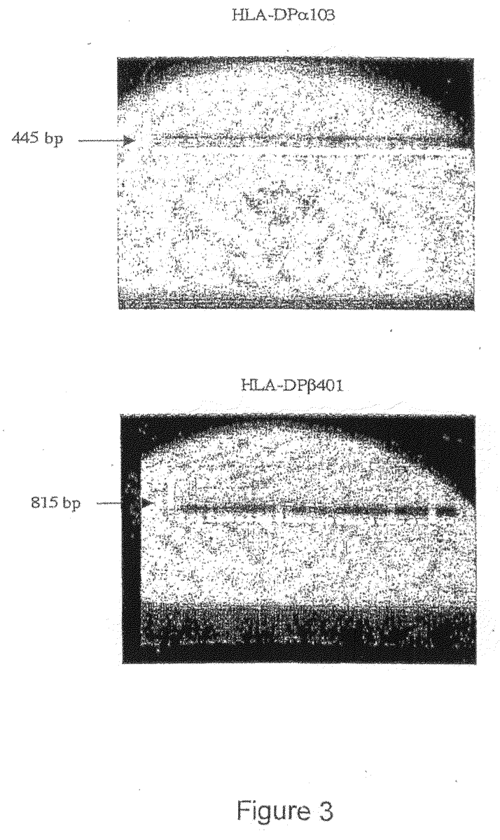

FIG. 3, the diagnostic PCR for HLA-DP transgenes;

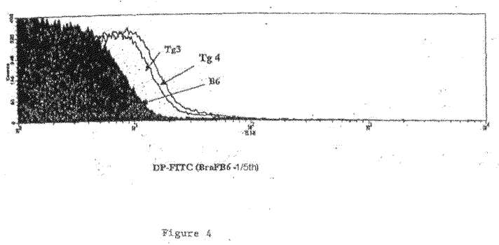

FIG. 4, the flow cytometry analysis of the expression of HLA-DP molecules in transgenic mice of the invention;

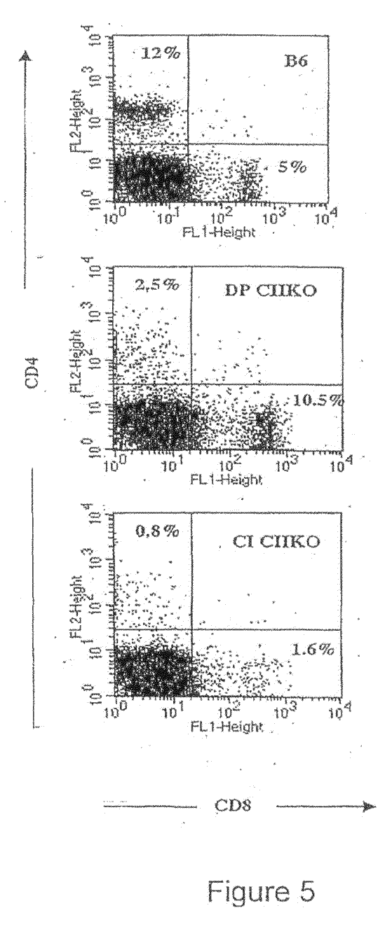

FIG. 5, the number of CD8+ and CD4+ spleen T cells;

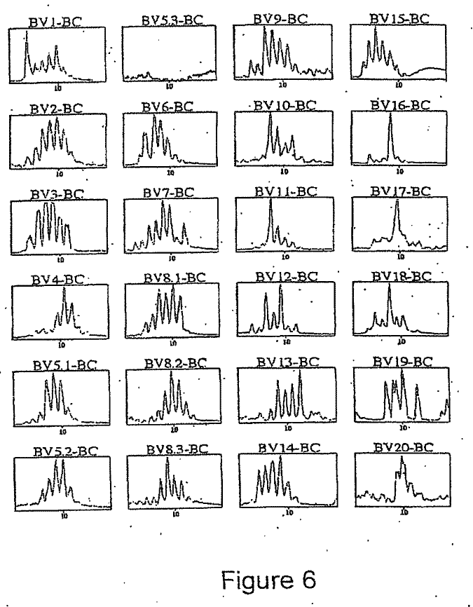

FIG. 6, the repertoire diversity of the various TCR Vb of the CD4+ T lymphocytes in the) (HLA-DP+/IAβ°) mice and C57BL6 mice;

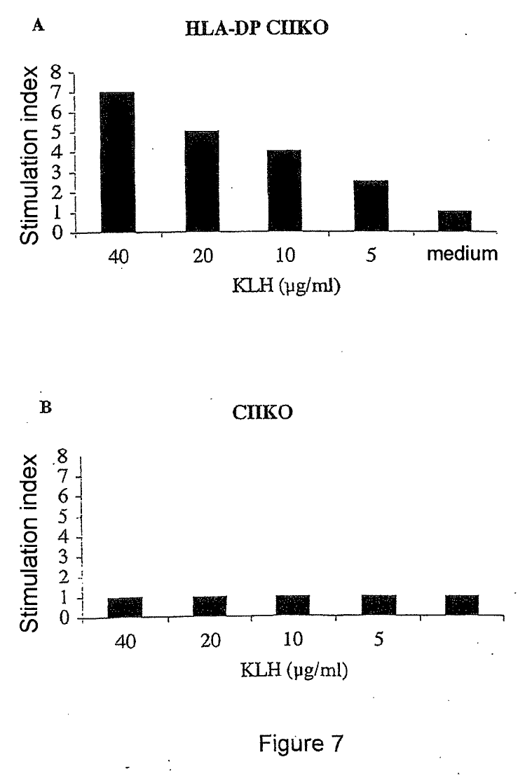

FIG. 7, the specific proliferative response after immunization with KLH of transgenic mice of the invention;

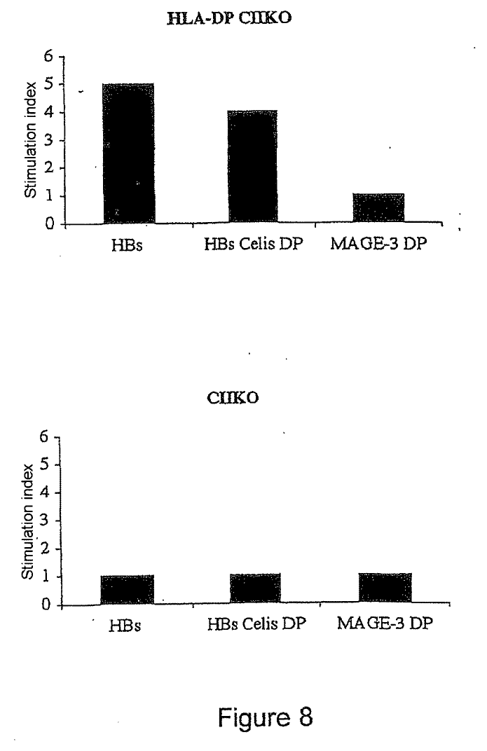

FIG. 8, the specific proliferative response after immunization with an HBs antigen of transgenic mice of the invention;

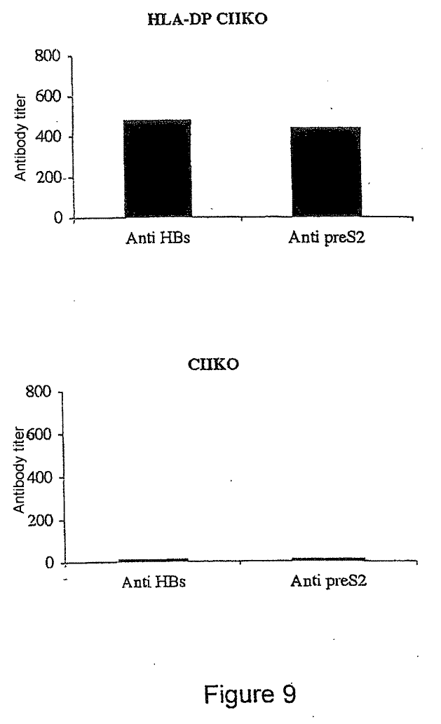

FIG. 9, the specific antibody responses after immunization with an HBs antigen of transgenic mice of the invention, and

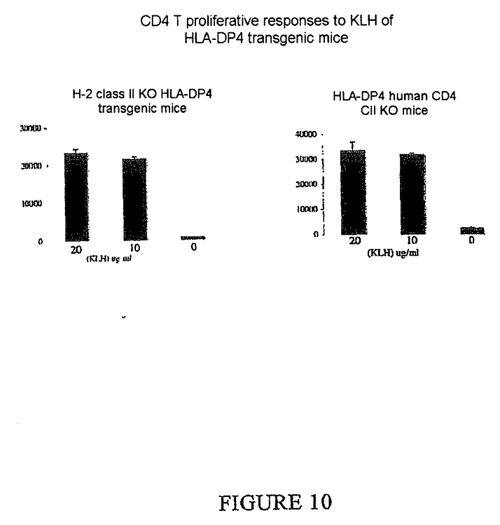

FIG. 10, the histograms represent a comparative study between the mice (HLA-DP(α103β401)IAβ° and HLA-DP(α103β401)IAβ°. hCD₄^{+/+}mCD₄°/°.) of the specific TCD4 proliferative response following an immunization using given antigens. The genetic background of the two mice corresponds to that of C57/BL6 mice.

### MATERIALS AND METHODS

1) Starting Genes and cDNA

IAβ b gene: 9 Kb KPNI-XBAI fragment isolated from the Aβ19 cosmid and in an entirely genomic configuration. The choice of the two restriction sites is based on the Lahrammar mapping (JBC, 1985, 260, 14111-14119).

IAαb gene produced by subcloning of a KpnI-SalI fragment taken from the LS8.1 cosmid (after KpnI trimming) of 6.8 Kb and ligated in 3′ to a 250 by SalI-NotI fragment taken from pUT 626 and providing the SV 40 polyadenylation site.

Obtaining of the cDNA for DPβ401 and DPα103

The total RNA was isolated from HOM-2 cells of homozygote HLA haplotype (A3, C1, B27, DR1, DQ5, DPβ0401, DPα0103), which are cells transformed with the Epstein Barr virus and expressing MHC class II molecules. RT-PCRs were carried out on these mRNAs using, as primers:

For DPβ401, 5′ primer (SEQ ID No. 1) 5′ CCT TTT ATC gAT CCA TgA Tgg TTC TgC Agg 3′ with introduction of a BspD1 site for subcloning in IAβ^{b};

3′ primer (SEQ ID No. 2) 5′ TTA AAA gTC gAC TTA CCT gTT TAT gCA gAT CCT C 3′ with the introduction of a Sal1 site for subcloning in IAβ^{b}.

For DPα103, 5′ primer (SEQ ID No. 3) 5′ CCT TTT ATC gAT CAA CAT gCg CCC TgA AgA C 3′ with introduction of a BspD1 site for subcloning in IAα^{b};

3′ primer (SEQ ID No. 4) 5′ TTA AAA CTC gAg gTg TgA gCA CgT ACC gTT ggT ggC CTg AgT gTg 3′ with the introduction of an Xho 1 site for subcloning in IAα^{b}.

Long forms DPαlong and DPβlong were prepared (whole of the complementary DNAs of DPα and of DPβ) and were introduced into pCR Topo 2 vectors (Invitrogen) and then cloned in RZ1032 bacteria).

2) Modification of the IAα^{b} and IAβ^{b} Genes by Site-Directed Mutagenesis

The site-directed mutageneses were carried out by the Kunkel method (P.N.A.S. USA, 1985 January; 82(2): 488-92) in order to introduce the restriction sites required for the cloning of the DPα DPβ cDNAs while respecting the various reading frames. Briefly, a single-stranded DNA template is generated by infecting, with a KO.7 bacteriophage, RZ1032 bacteria (UTPase⁻, incapable of removing the Us incorporated by error) transfected beforehand with vectors containing the target genes. The mutagenesis oligonucleotide, which has been phosphorylated, is hybridized on this template. A DNA polymerase synthesizes the mutated strand. The transfection of supercompetent XL1(UTPase⁺) bacteria is then carried out, in which bacteria the substituted U strand (not mutated) is degraded and the mutated strand is conserved.

Long Forms

DPβ401long: subcloning of the EcoR1 fragment (signal sequence and sequences of the human β1 and (β2 domains) of pcR2 Topo into a pBlueScript/KS⁻ plasmid containing the HindIII-Xba1 fragment (3 kb) of IAβ^{b} BspD1+Sal1+. The BspD1-Xba1 fragment obtained from this construct was inserted into pBlueScript/KS⁻IAβ^{b} opened with BspD1-Xba1. The end portion of IAβ^{b} was thus reconstituted and complete DPβ401 is inserted.

DPα103long: subcloning of the BspD1-Sal1 fragment (signal sequence and sequences of the human α1 and α2 domains) of pcR Topo 2 into pBlueScript/KS⁻IAα^{b} opened with BspD1-Sal1.

The DPβ401long form was subsequently introduced into a vector containing a hygromycin cassette so as to give the final construct pIADPβlong (8.5 Kb) (FIG. 1).

The DPα103long form was introduced into a vector containing a neomycin cassette so as to give the final construct pIADPαlong (8 Kb) (FIG. 1). These various constructs were subsequently cloned in the supercompetent XL1 bacteria and the sure bacteria (rendered competent), and then verified by sequencing by the Sanger technique.

Introduction of a Strong Rous Sarcoma Promoter in Front of the Long DPβα Constructs

pSRDPαlong (7.9 kb): subcloning of the SacI-EcoRV fragment of DPαlong-pcR2 Topo into NT Hygrod opened with SmaI(blunt)-SacI.

pSRDPβ1g (7.7 kb): subcloning of the SacI-EcoRV(blunt) fragment of DPβlong corrected c.8-15 pcR2 Topo into NT Neod opened with SacI-EcoRV.

3) Cells and Transfectants

HeLa cells (human uterine cervix cells), L Kuhn cells (C3H H-2^{k} mouse fibroblasts) and P815 cells (mouse mastocytoma originating from a DBA/2 H-2^{d} mouse) were maintained in complete RPMI 1640 medium: 10% FCS, penicillin-streptomycin (200 IU/ml), sodium pyruvate (1 mM) and L-glutamine (2 mM). Cells in the exponential growth phase were cotransfected with the long forms+/−CIITA (transcriptional regulation factor for MHC II genes); 24 h after the transfection, they were selected with G418 (1 mg/ml) and then 24 h later with hygromycin B (400 μg/ml).

The clones obtained were tested by direct immunofluorescence and by flow cytometry (FACScan, Becton Dickinson). Briefly, 500 000 cells are incubated for 40 minutes with a murine antibody which recognizes HLA-DP (B721), IA^{d} (MKD6) or HLA-A3 (GAPA 3), in PBS 1% BSA 2 mM EDTA, and then, after washing, are incubated for 40 minutes with an FITC-coupled goat anti-mouse second antibody, in PBS 1% BSA 2 mM EDTA, and washed in PBS BSA 2 mM EDTA.

4) Genomic DNA Extraction, Southern Blotting Analysis and Diagnostic PCRs for the Transgenes

The genomic DNA is prepared from transfectants or from mouse tails according to the following method: treatment with proteinase K, incubation for 16 h at 56° C., treatment with saturated NaCl, precipitation of the DNA with 2-isopropanol, washes with 70% ethanol and DNA pellet taken up in 150 ul of 10 mM Tris 1 mM EDTA, pH 7.5. DNAs extracted from the DP transfectants were digested with BamHI for the Southern analysis diagnosis of DPαlong and with EcoRI for the DPβlong diagnosis, and then, after 0.6% agarose gel electrophoresis, were blotted onto a nitrocellulose membrane and hybridized with their corresponding probe. The DPα probe corresponds to the complete DPα103 cDNA, the DPβ probe is the DPβ401 cDNA. These probes were radiolabeled with dCTP32 according to the manufacturer's guidelines (Megaprime DNA labeling system, Amersham Pharmacia).

The expression of the DPβ401 and DPα103 transgenes was detected by PCR using primers specific for the DP sequences. The PCR was carried out in tubes containing mouse genomic DNA (300 ng for DPβ401 and DPα103), PCR buffer, 15 pM of each primer, 12 pM of each dNTP and 1.25 U of Taq polymerase (GibcoBRL) with a final concentration of MgCl2 of 2.6 mM for DPβ401 and of 4.1 mM for DPα103. A 1st denaturation cycle is carried out for 7′ at 94° C. followed by 30 cycles consisting of 30″ of denaturation at 94° C., 30″ of hybridization at 55° C. and 30″ of extension at 72° C., and a final extension cycle for 4′ at 72° C.

For DPβ401, the sense primer is 5′ (SEQ ID No. 5) ggATTggAAAgAggCTC 3′ and the antisense primer is 5′ (SEQ ID No. 6) gCACtgCCCgCTTCTCC 3′. For DPα103, the sense primer is 5′ (SEQ ID No. 7) TAATACAAAgTCTgCAgCTggC 3′ and the antisense primer (SEQ ID No. 8) is 5′ AgCAATgTTAgCCAgCC 3′.

Construction of Expression Vectors Encoding the HLA-DPα103 or HLA-Dβ401 Transgene

The maps of the 4 plasmids pIADPαlong, pIADPβlong, pSRDPαlong and pSRDPβlong are reported in FIGS. 1A to 1D, respectively.
pIADPαlong: promoter IAα/DPα103/NcIAα
pIADPβlong: promoter IAP/DPβ401/NcIAβ
pSRDPβalong: promoter SR/DPα103/NcSV40
pSRDPβlong: promoter SR/DPβ401/NcSV40.

The plasmids are entirely sequenced and verified.

Construct Functionality

It was assessed by transfection of murine fibroblasts (L Kuhn, H-2k) and of mouse mastocytomas (P815, H-2d). These two cell types do not naturally express class II histocompatibility molecules. The transfection of the DPβ DPα genes was combined with that of a plasmid encoding the human transcriptional regulatory factor CIITA, which determines the activation of the HLA class II genes (α and β) of the HLA-DP, -DQ and -DR series and also activates the expression of the gene encoding the invariable chain. The MKD6 (anti-IAd) and GAPA3 (anti-HLA-A3) antibodies having the same immunoglobulin isotype as the B721 antibody were used as controls. The surface expression of the wild-type DPαβ constructs could once again be documented at the surface of L transfectants (not reactive with the anti-IAd MKD6) and of P815 transfectants. In the latter case, a strong reactivity was observed with the MKD6 antibody but not with GAPA3, reflecting the transcriptional activity of the IAd genes subsequent to the transfection of the CIITA gene.

The functionalities of the pSRDPαlong and pSRDPβlong vectors were assessed by cotransfection of L Kuhn human cells and of HeLa cells. Those of the pIADPαlong and pIADPβlong vectors were assessed by cotransfection in addition to the pCIITA plasmid and human CIITA (human CHTA transcriptional regulation factor) of P815 cells.

The results obtained are illustrated in FIG. 2. FIG. 2A corresponds to the P815 nontransfected HLA-DP transfectants, transfectants cotransfected with Dpαlong/Dpβlong/CIITA or with CIITA, incubated with the monoclonal antibody B721 (anti-HLA-DP), MKD6 (anti-IA) or GAPA3 (anti-HLA-A3), and then with a goat anti-mouse IgG-FITC. The cells are analyzed by flow cytometry. FIG. 2B corresponds to the L-Kuhn-DP and Hela-DP transfectants.

These assessments made it possible to verify that these constructs are functional and encode the functional HLA-DPα103β401 complex recognized specifically by specific anti-HLA-DP antibodies.

Creation of Transgenic Mice and Diagnostic PCRs for the Transgenes

Two transgenesis approaches were carried out:

a series of single transgeneses was carried out at the Paul Brousse hospital:

the DPαlong and DPβlong constructs were microinjected individually into fertilized eggs of C57BL/6×DBA2 mice;
a series of double transgeneses was carried out at the Cochin hospital by microinjection of the DPαlong/DBβlong constructs into fertilized eggs of C57BL/6×DBA2 mice.

In order to detect the single or double transgenic founder mice, a diagnostic PCR test was developed and carried out on DNA extracted from mouse tails. FIG. 3a shows the result obtained and the detection of the DPalong transgenes. A band of 445 by characteristic of this transgene appears with the DNAs of the mice transgenic for DPαlong. This band is naturally absent with the DNAs of the DBA2 and B6 control mice. FIG. 3b illustrates the detection of the DPβlong transgenes. The band of 815 by obtained is specific for these transgenes.

11 transgenic DP mice were obtained;

5 DPαlong mice

3 DPβlong mice.

Founder mice expressing individually the HLA-DPα103 or HLA-DPβ401 transgene were crossed with one another. The mice expressing simultaneously the two transgenes HLA-DPα103 and HLA-DPβ401 were then crossed with C57BL/6IAβ° mice in order to obtain HLA-DPα103β401+IAβ° mice.

The expression of various transgenes or the absence of expression of the IAβ gene are analyzed by PCR and some of them are also analyzed by Southern blotting.

Protocol for the Diagnostic PCRs for the Transgenes:—HLA-DPα103

Products: TAQ DNA polymerase (Invitrogen)

50 mM MgClII (Invitrogen)

10× PCR buffer (Invitrogen)

sterile H₂O

dNTP 4 mM for each

Primer:

promoter IAα (sense primer) (SEQ ID No. 9)
5′ TAA TAC AAA gTC TgC AgC Tgg C 3′
DP2α antisense (SEQ ID No. 10)
5′ AgC AAT gTT AgC CAg CC 3′

(MgCl₂) final concentration=4.1 mM

Tm 55° C.

For DPα, a fragment characteristic of the transgene, of approximately 445 bp, is thus amplified. The B6 and DBA/2 negative controls do not exhibit any amplification.

PCR cycles 94° C. 7 minutes
(94° C. 30 sec, 55° C. 30 sec, 72° C. 30 sec)
×40 cycles
72° C. 4 minutes
4° C.

HLA-Dβ401 Transgene Diagnostic PCR

Products: TAQ DNA polymerase (Invitrogen)

50 mM MgCl₂ (Invitrogen)

10× PCR buffer (Invitrogen)

sterile H₂O

dNTP 4 mM for each

Primer:

promoter IAβ (sense primer) (SEQ ID No. 11)
5′ ggA TTg gAA AgA ggC TC 3′
DPβ antisense (SEQ ID No. 12)
5′ gCA CTg CCC gCT TCT CC 3′

(MgCl₂) final concentration=2.6 mM

Tm 55° C.

For DPβ, a fragment characteristic of the transgene, of approximately 815 bp, is thus amplified. The B6 and DBA/2 negative controls do not exhibit any amplification.

PCR cycles 94° C. 7 minutes
(94° C. 30 sec, 55° C. 30 sec, 72° C. 30 sec)
×40 cycles
72° C. 4 minutes
4° C.

Phenotyping of the HLA-DPα103β401+/IAβ° Mice

The level of cell surface expression of the HLA-DP transgenic complexes in the HLA-DP+/IAβ° transgenic mice was measured by flow cytometry. The results are given in FIG. 4.

The number of peripheral CD4+ T lymphocytes and of CD8+ cells was measured in the mice (HLA-DP+Iab°). FIG. 5 illustrates the results obtained.

The splenocytes of C57BL/6 mice (B6 in FIG. 5A), HLA-DP/H-2 class II-KO mice (DPCHKO in FIG. 5B) and H-2 class I/class II-KO mice (CI CHKO in FIG. 5C) were stained with CT-CD4 monoclonal antibodies labeled with PE (mouse anti-CD4 antibody, along the y-axis) and antibodies labeled with FITC 53-6,7 (anti-CD8 antibody, along the x-axis). The numerical values indicated correspond to the CD4+ T cells (FIG. 5A, upper left part) or to the CD8+ T cells (FIG. 5C, lower left part) in the total splenocytes.

By using the immunoscope technology, it was shown that the repertoire diversity of the various TCR Vbs of the CD4+ T lymphocytes is comparable between the (HLA-DP+/IAβ+) mice and the C57BL6 mice (see FIG. 6).

Functional Study

By using the KLH antibody (T dependent), it was shown, in 6/6 (HLa-DP+/IAβ°/°) mice, that they were capable of a proliferative response to this antigen, whereas no response was observed in 6/6 (IAβ°/°) mice. The results obtained are illustrated in FIGS. 7A and 7B.

Other experiments consisted in using the HBs envelope antigen (preS2-S) of the hepatitis B virus as a study model for simultaneously analyzing the anti-HBs cellular and humoral response between the (HLA-DP+/IAβ°/°) mice, subsequent to a genetic immunization by injection of the pCMV-HBs plasmid encoding the HBs protein (preS2-S).

The following assays were used:
ELISA assay for monitoring the specific anti-HBs humoral response (anti-preS2 and anti-S antibodies);
specific cell proliferation assay for monitoring the helper cell response specific for the HLA-DP restricted epitopes derived from the HBs protein (HBs Celis DP).

The HLA-DP-restricted T epitope HBs Celis DP (FLLRILTIP) is described in E. Celis and R W Karr, J. Virol. 1989, February; 63(2): 747-52.

The presence, simultaneously, of a strong specific proliferative cell response (proliferative index >3) for the HBs Celis DP T epitope (see FIG. 8) and the presence of anti-HBs and anti-pre-S2 antibodies (see FIG. 9) were documented in 3/4 (HLA-DP+/IAβ°/°) mice immunized against HBs.

No IAβ° mouse (0/4) immunized with the pCMV-S2S plasmid gives a specific cellular response for the HBs T epitope, or an anti-preS2 or anti-HBs antibody response.

These results show that the (HLA-DP+/IAβ°/°) mice represent a novel animal experimental model of humanized mice which is optimal for the identification of the HLA-DP restricted epitopes present in the various antigens.

HLA-DP(α103β401)IAβ°/° hCD₄^{+/+}mCD₄/Transgenic Mice

Reported hereinafter are the results of experiments carried out with H-2 class II KO transgenic HLA-DP4 mice (HLA-DP(α103β401)IAβ°/°) and with HLA-DP4 human CD4 CII KO mice (HLA-DP(α103β401) IAβ°/° hCD₄^{+/+} mCD₄°/°, relating to the study of the CD4 T proliferative responses after immunization with
HLA-DP4 peptide (Celis)

### CD4 T Proliferative Responses of Transgenic HLA-DP Mice, Immunized with Ii-DP (Celis), to the HLA-DP4 Peptide (Celis)

| H-2 class II KO transgenic | HLA-DP4 human CD4 CII KO |
|---|---|
| HLA-DP4 mice | mice |
| 2.4 | 3.7 |
| 1.5 | 1.4 |
| 2.1 | 2.3 |
| 2.1 | 2.8 |
| 1.5 | 3 |
| 2.2 | 2.8 |
| 2.3 | 2.5 |
| | 2.3 |

3% of CD4 T lymphocytes is observed in the H-2 class II KO transgenic HLA-DP4 mice versus 0.8% in the H-2 class II KO mice, and 12% of CD4 T lymphocytes in the HLA-DP4 human CD4 murine CD4 KO CII KO transgenic mice.

The CD4 T proliferative responses to KLH of transgenic HLA-DP4 mice are illustrated by the histograms of FIG. 10A (H-2 class II KO HLA-DP4 transgenic mice) (HLA-DP(α103β401) IAβ°/°) and 10B (HLA-DP4 human CD4 CII KO mice) (HLA-DP(α103β401) IAβ°/° hCd₄^{+/+} mCD₄°/°).

1. The use, for creating transgenic mice, of an expression vector construct encoding the functional HLA-DPα103β401 complex, specifically recognized by anti-HLA-DP antibodies, characterized in that the construct comprises murine promoters of the IAβ and IAα genes associating the cDNAs encoding DPα103 and DPβ401.

2. A transgenic mouse as obtained according to claim 1, characterized in that it is a humanized mouse expressing individually the HLA-DPα103 or HLA-DPβ401 transgene or simultaneously the 2 transgenes.

3. The mouse as claimed in claim 2, transgenic for HLA-DP(α103β401) and knockout for H-2 class II (HLA-DP+/+IAβ°/°).

4. The mouse as claimed in claim 3, transgenic for HLA-DP(α103β401) hCD4+/+ and knockout for H-2 class II IAβ°/° mCD4°/°.

5. The use of the mice as claimed in claim 3, for the comparative preclinical study of the efficacy of vaccine candidates.

6. The use of the mice as claimed in claim 3, for assessing the risks associated with an unwanted induction of an autoimmune disease.

7. The use of the mice as claimed in claim 3, for determining a therapeutic strategy.

8. The use of the mice as claimed in claim 3, for identifying new epitopes which enable the development of reagents for monitoring the specific immune response post-infection or post-immunization.

9. A CD4+ lymphocyte isolated from mice as claimed in claim 3, after immunization of the latter.

10. The use of the CD4+ lymphocyte as claimed in claim 9, for establishing specific cell lines.

11. The use of the CD4+ lymphocyte as claimed in claim 9, for creating specific T hybridomas by cell fusion.

12. The cell lines established as claimed in claim 10.

13. The hybridomas obtained as claimed in claim 11.
